(19) United States
(12) Patent Application Publication
(10) Pub. No.: US 2016/0256392 A1
(43) Pub. Date: Sep. 8, 2016

(54) ABUSE-DETERRENT DOSAGE FORMS

(71) Applicant: CIMA LABS INC., Brooklyn Park, MN (US)

(72) Inventors: Dinesh K. HASWANI, Plymouth, MN (US); Derek V MOE, Mound, MN (US); Victoria A. O'NEILL, Wayzata, MN (US); Manuel A. VEGA ZEPEDA, Minnetonka, MN (US)

(21) Appl. No.: 15/033,119

(22) PCT Filed: Jul. 17, 2014
(86) PCT No.: PCT/US2014/046984

§ 371 Date: Apr. 29, 2016

Related U.S. Application Data
(60) Provisional application No. 61/898,214, filed on Oct. 31, 2013.

(51) Int. Cl. A61K 9/20; A61K 31/167; A61K 31/485

(52) U.S. Cl. CPC A61K 9/2009; A61K 31/485; A61K 9/2013; A61K 9/2018; A61K 9/2054; A61K 9/2027; A61K 31/167; A61K 9/2031; A61K 9/205

(57) ABSTRACT

Described are oral dosage forms that contain abuse-deterrent features and that contain a matrix with gelling polymer and disintegrant, with particular examples including immediate release dosage forms that contain a drug that is commonly susceptible to abuse.

### FIELD OF THE INVENTION

The present invention relates to the field of oral dosage forms that contain abuse-deterrent features, in particular including immediate release dosage forms that contain a drug that is commonly susceptible to abuse.

### BACKGROUND

Pharmaceutical products, including both prescription and over-the-counter pharmaceutical products, while useful for improving health of a person in need, are also susceptible to intentional and unintentional abuse and overdosing. Examples of commonly abused active pharmaceutical ingredients include psychoactive drugs, anxiolytics, sedative hypnotics, stimulants, depressants, and analgesics such as narcotic analgesics, among others. A complete list of specific drug compounds that are commonly abused would be lengthy; a short listing of some classes of drugs commonly abused includes opioids and morphine derivatives, barbiturates, amphetamines, ketamine, and other drugs that can cause psychological or physic al dependence.

Some common techniques for intentionally abusing a drug begin with an abuser obtaining a solid dosage form such as an orally administered tablet or capsule, and crushing the solid dosage form into a powder. The powder may be administered by an abuser by nasal insufflation (i.e., “snorting”) to introduce the drug to the abuser's bloodstream intranasally. Alternately, the crushed dosage form may be combined with a solvent that is capable of dissolving the drug (active pharmaceutical ingredient, or “API”), and the solvent with the dissolved drug may be injected directly into an abuser's bloodstream.

Alternatively, with immediate release oral dosage forms, an abuser might simply ingest multiple units (e.g., tablets) of the dosage form together, e.g., simultaneously. Each one of the multiple dosage form units would immediately release an amount of drug to produce a short-term concentration spike of the drug in the user's bloodstream and a desired “high” in the user.

The pharmaceutical industry has identified various mechanisms of adapting drug compositions and oral dosage forms that can be useful to discourage abuse of oral dosage forms. Pharmaceutical companies have studied dosage forms with an added nasal irritant or an added effervescent agent, which can cause irritation or pain in a nasal passage if the dosage form is crushed and then snorted, thus discouraging abuse by nasal insufflation. Pharmaceutical companies have also studied adding gelling polymers to dosage forms to prevent abuse by injection. If the dosage form is crushed to a powder and combined with a small amount of solvent, the gelling polymer can cause the combination to take the form of a highly viscous liquid or gel that cannot be administered by injection. Another possible abuse deterrent may be addition of an emetic agent which can deter abuse by causing emesis on ingestion ?if multiple doses are ingested. Another abuse deterrent involves adding an antagonist of an API to a dosage form that will substantially block the effect of that API.

Although the pharmaceutical industry has identified of a variety of abuse deterrent features useful with oral dosage forms, there is continuing need to improve and identify new abuse deterrent features to inhibit or prevent abuse or overdosing of active pharmaceutical ingredients.

### SUMMARY

The following description relates to oral dosage forms that are useful for immediate release of an active pharmaceutical ingredient or “API.”

The dosage form can be designed to release the API as desired in an immediate release dosage form. At the same time, the dosage form can be resistant to abuse. Exemplary dosage forms can include a gelling polymer that functions as an abuse deterrent feature by compromising abuse practices wherein the dosage form is crushed and then combined with a small amount of a solvent to produce a liquid composition that contains a concentrated amount of API and that can be delivered to an abuser using a syringe. Preferred gelling polymers are carbomers (poly acrylic acid polymers) and xanthan gum.

These gelling polymers have previously been used in extended release (ER) dosage forms to effectively slow down the release of an API from the extended release dosage form. The gelling polymers are known to inhibit or retard release of API from a dosage form. According to the present description, however, Applicant has now identified that these gelling polymers can be incorporated into a dosage form in a manner whereby the gelling polymer functions as an abuse deterrent feature without unduly inhibiting or retarding release of the API.

The dosage form also includes filler, as well as a disintegrant. As explained supra, examples of the described dosage forms include an amount of gelling polymer that can be sufficiently high to allow the gelling polymer to prevent uptake of a dissolved ground dosage form. To overcome the potential gelling polymer effect of retarding release of the API during ingestion for an intended therapeutic use, dosage forms as described include a disintegrant, the amount of the disintegrant being sufficient to provide desired immediate release of the API upon such ingestion.

The active pharmaceutical ingredient included in the dosage form can be any active pharmaceutical ingredient desired to be administered orally, and may in particular be a type of active pharmaceutical ingredient that is commonly susceptible to abuse. Examples of active pharmaceutical ingredients that are considered to be commonly susceptible to abuse include psychoactive drugs, tranquilizers, sedative hypnotics, anxiolytics, stimulants, depressants, and narcotic analgesics, among others. Certain more specific classes of drugs commonly abused includes opioids, barbiturates, benzodiadepines, amphetamines, as wells as other drugs that are known to cause psychological or physical dependence.

Dosage forms of the present description can be useful as immediate release dosage forms, and also generally include abuse deterrent features such as features that discourage or prevent abuse by nasal insufflation or by injection. More particularly, certain embodiments of the described dosage forms that include gelling polymer comprising carbomers (poly acrylic acid polymers) and xanthan gum, or a combination thereof, have been shown to provide particularly effective extraction resistance, meaning resistance to extraction of the API into a solvent for uptake in a syringe as is commonly done in methods of drug abuse. For example, embodiments of dosage forms that include at least 5 weight percent carbomer as a gelling polymer exhibit a high level of extraction resistance, preventing extraction of a useful amount of API from a crushed and dissolved dosage form for subsequent injection using a hypodermic needle and syringe. Likewise, embodiments of dosage forms that include at least 2 or 3 weight percent xanthan gum as a gelling polymer also exhibit a high level of extraction resistance. And, as referenced supra, Applicant has also determined that even though these described dosage forms form viscous gels and prevent uptake in a syringe, the same dosage form can be made to provide the desired immediate release of API by including a sufficiently high level of disintegrant.

Embodiments of the described dosage forms can be effective in the absence of other types of abuse deterrent features such as nasal irritants, emetic agents, bittering agents, and effervescents, to inhibit nasal insufflation or other forms of abuse, or the inclusion of drug antagonists of the subject drug.

In one aspect, the invention relates to an immediate release compressed oral dosage form. The dosage form includes: an active pharmaceutical ingredient; from 2.5 to 35 weight percent gelling polymer comprising a carbomer polymer; from 15 to 35 weight percent disintegrant; from 3 to 80 weight percent filler, and a pH adjuster; the weight percent amounts being based on a total weight of the dosage form.

In another aspect the invention relates to an immediate release compressed oral dosage form that includes: an active pharmaceutical ingredient; from 1 to 20 weight percent gelling polymer comprising xanthan gum; from 15 to 35 weight percent disintegrant; and from 3 to 80 weight percent filler; the weight percent amounts being based on a total weight of the dosage form.

### BRIEF DESCRIPTION OF THE DRAWINGS

FIG. 1 shows data for simulated extraction of hydrocodone tartrate from crushed tablets manufactured using direct compression processes according to Examples 22, 24 and 38.

FIG. 2 shows immediate release profiles of dosage forms manufactured using direct compression processes according to Examples 22, 24, 28 and 38.

### DETAILED DESCRIPTION

The present description relates to immediate release dosage forms that include one or more abuse deterrent features for reducing the potential for parenteral abuse and abuse by nasal insufflation. These abuse deterrent features are achieved by combining certain ingredients into a matrix of a compressed dosage form. The combinations of ingredients in a compressed dosage form have now been determined to effectively prevent an abuser from realizing the intended biological effect of the drug abuse by using certain presently-common methods used to abuse the API. Advantageously, a dosage form prepared to contain one or more of the described abuse deterrent features, as a deterrent to abuse of one or more API that is commonly susceptible to abuse, can still be constructed to provide immediate release of the one or more API upon normal therapeutic use by oral ingestion.

The term “immediate release” refers to a dosage form that upon oral ingestion by a human releases substantially all of a contained active pharmaceutical ingredient into a gastrointestinal tract for biological uptake in a short time. In vitro methods of measuring a release profile of a dosage form, for the purpose of determining whether a dosage form exhibits an immediate release or dissolution profile, are known in the pharmaceutical arts. By such methods, examples of dosage forms as described herein can be measured to be capable of releasing substantially all of a total amount of at least one type of active pharmaceutical ingredient (e.g., an API commonly susceptible to abuse) contained in the dosage form (e.g., at least 75, 80, or 90 weight percent of the total amount of the API in a dosage form) into a solution (e.g., acidic aqueous solution) of a suitable pH within 240 minutes, e.g., in less than 180 minutes, less than 90 minutes, or less than 60, 30, 15, or 5 minutes. For example, a release profile of a dosage form of the present description may be measured by a method that exposes the dosage form to a volume of up to 900 milliliters (e.g., 300 milliliters, or 900 milliliters, based on various test methods) of hydrochloric acid (0.01 to 0.1N) (e.g., aqueous hydrochloric acid) at a pH of from 1 to 2, and at a temperature of 37 degrees Celsius.

Dosage forms as described can be formulated to provide an immediate release profile of an API, and can also be prepared to include effective or advantageous abuse deterrent features that are effective to deter abuse of the same API (e.g., one that is commonly susceptible to abuse) that exhibits the immediate release profile. The described dosage forms, which provide a combination of immediate release of an API, with broad abuse resistance for the same API, including highly effective uptake resistance, is not believed to be previously known. More particularly, dosage forms as described can provide an immediate release profile of an API, and can at the same time include abuse deterrent features that provide general abuse deterrence of the same API. The dosage forms can also be more specifically characterized as resistant to certain common methods of abuse, such as abuse by injection (e.g., by steps that include grinding a dosage form and dissolving API of the dosage form) and abuse by nasal insufflation (e.g., also by grinding and optionally dissolving API of a dosage form).

According to certain methods of drug abuse by injection or nasal insufflation, a dosage form may be ground and dissolved in a “small volume” of solvent, and then taken up by (or “extracted”) using a hypodermic syringe for abuse by injection or nasal insufflation. The solvent is one commonly available to and useful to an abuser, such as water and C₁-C₄ alcohols (e.g., ethanol and methanol). A “small volume” refers to an amount of such a solvent that can contain an amount of dissolved API that is sufficiently concentrated to be useful to an abuser to realize the intended biological effect of the drug abuse, and that is also capable of being administered for abuse of the API, e.g., a volume that can contain an amount (concentration) of API that is effective to achieve a desired “high” if administered by injection or nasal insufflation, the volume also being sufficiently small to allow the volume to be administered by injection or nasal insufflation. For a dosage form to be useful for abuse as such, an API in the dosage form must be capable of being accessed and dissolved at sufficient concentration by an abuser without undue complication, into a “small volume” of solvent, which is a volume that can be administered by injection or by nasal insufflation. Generally, a “small volume” of solvent means 50 milliliters or less, or 20 milliliters or less, or 10 milliliters or less, or 5 milliliters or less (volumes which could be injected or used for nasal insufflation).

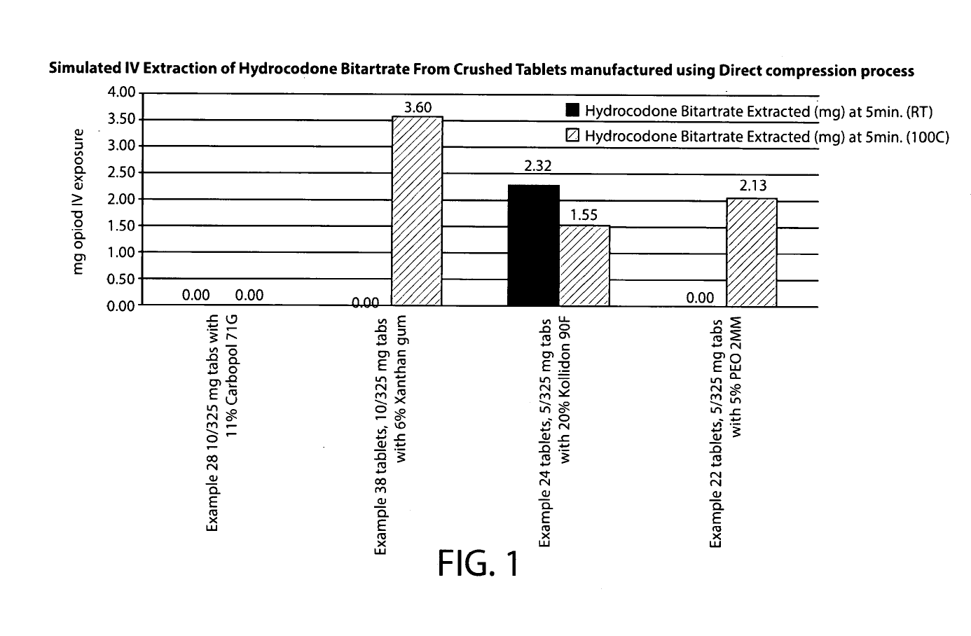

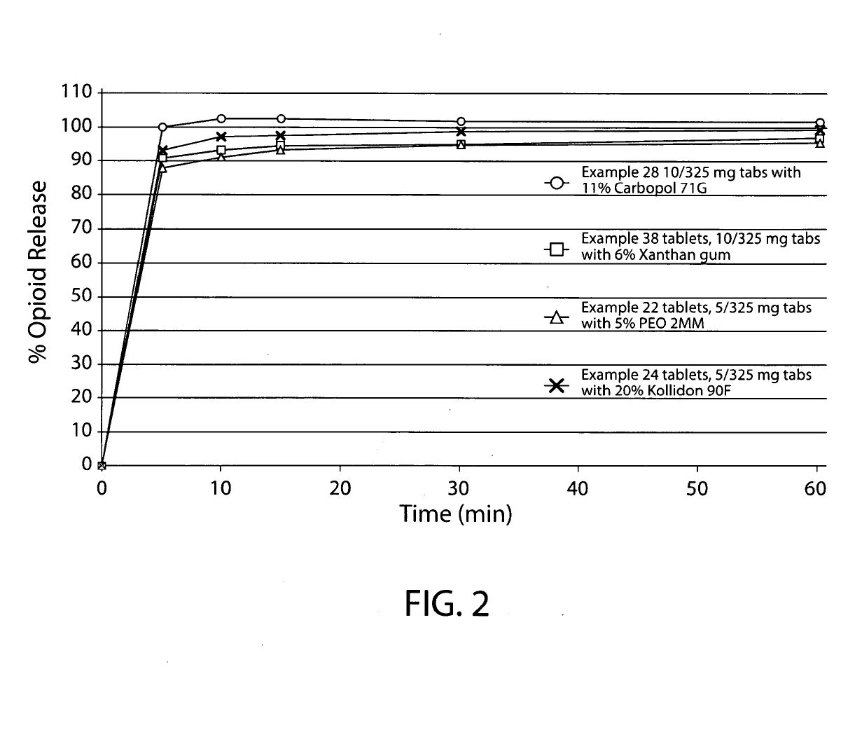

To prevent abuse by methods that include an abuser dissolving the API in a solvent, as described, and administration using a syringe, a dosage form may be adapted to prevent the API from being accessed by being dissolved in a small volume of a commonly-used solvent, i.e., may exhibit “extraction resistance.” Certain examples of the dosage forms as described can exhibit particularly strong extraction resistance. See FIG. 1. (FIG. 2 shows immediate release profiles of example dosage forms.)

Testing for extraction resistance of an oral dosage form can be performed by known methods of dissolving a dosage form at specified conditions in a commonly available solvent (e.g., water, or a C₁₋₄ alcohol such as ethanol or methanol) and attempting to generate a solution of the API for injection using a hypodermic needle and syringe. Dosage forms of the present description, and as specified at FIG. 1, were subjected to simulated intravenous (IV) isolation testing as both intact and crushed tablets. The tablets were placed in small volume 10 milliliters of water for up to 5 minutes at room temperature (about 25° C.) and at 100° C. The resultant combined dosage form and water material was assessed for its ability to be syringed through a filter material for intravenous administration. The filtrate was then analyzed for the amount of active pharmaceutical ingredient (hydrocodone bitartrate) that was extracted.

The results at FIG. 1 show that a dosage form as described he rein (Example 28), containing 11% Carbopol® 71 G and 25 weight percent disintegrant, upon being combined with a small volume of water, formed a viscous gel that could not be drawn into a syringe, preventing any uptake of the API into the syringe. Effective resistance to uptake is considered to be achieved if the amount (mg) of measured API IV exposure is less than 1.00, e.g., less than 0.50, or less than 0.10, meaning that uptake is considered to be prevented. (“API IV exposure” refers to the amount of the API that is drawn into the syringe.) Alternately stated, effective resistance to uptake is considered to be achieved if the amount (percent) of API that can be drawn into a syringe is less than 50 percent of the total amount of API in the dosage form, e.g., less than 40 percent, 30 percent, 20 percent, 10 percent, or 5 or 1 percent.

A second embodiment of dosage form as described herein, containing 6 weight percent xanthan gum and 25 weight percent disintegrant (Example 38), upon being combined with a small volume of water, also formed a viscous gel that could not be drawn into a syringe, preventing any uptake of the API into the syringe.

As one type of abuse deterrent feature, a dosage form as described can include one or more gelling polymers. A gelling polymer can act as an abuse deterrent feature by preventing an active pharmaceutical ingredient of a dosage form from being dissolved in a small volume of solvent or being accessible or easily isolatable if combined with solvent with the gelling polymer also present. A gelling polymer can also deter or prevent abuse of an API in a dosage form by increasing the viscosity of a combination of the ground dosage form with solvent (especially a “small volume” of solvent) to a viscosity that is sufficiently high to prevent the combination or the API from being taken up by and injected using a syringe. A preferred gelling polymer contained in a ground dosage form, when exposed to a limited volume (or “small volume”) of solvent such as a C₁₋₄ alcohol (e.g., ethanol or methanol) or water, can form a non-injectable mass ranging from an insoluble mass, to a gel, to a viscous slurry, each of which exhibits a viscosity that prevents either uptake by or injection from a needle of a hypodermic syringe.

Suitable gelling polymers include one or a combination of polymers that, as part of a dosage form, upon contact of the dosage form with a small volume of solvent, will absorb the solvent and swell to form a viscous or semi-viscous substance that significantly reduces or minimizes the amount of free solvent that can contain an amount of a solubilized API and that can be drawn into a syringe. The gelled polymer can also reduce the overall amount of drug extractable with the solvent by entrapping the drug in a gel matrix.

The gelling polymer can be present in the dosage form at a location and in an amount that together allow the gelling polymer to produce a viscous gel in the event of an abuser grinding the dosage form and combining the crushed dosage form with a solvent. On the other hand, the gelling polymer, as present in the dosage form, will preferably not interfere with desired dissolution of the dosage form, the desired release (immediate release) of API from the dosage form, or the uptake of the API by a patient ingesting the intact immediate release dosage form for an intended therapeutic purpose. An exemplary location for the gelling polymer is as a component of a compressed matrix of a dosage form such as a compressed tablet or a compressed capsule.

The gelling polymer can be present in a dosage form at any desired amount and at any portion of, or location in a dosage form structure. The amount of gelling polymer can be any useful amount, meaning an amount that can produce an abuse-resistant viscous mixture or gel if the dosage form is crushed, ground, powdered, etc., and mixed with a commonly available solvent. A useful amount of total gelling polymer in a dosage form may be in a range from 0.5 to 50 weight percent gelling polymer based on a total weight of the dosage form, e.g., from 0.5 to 30 or from 1 to 40 weight percent gelling polymer based on total weight dosage form.

While these amounts can generally be useful for any single gelling polymer or for any combination of two or more gelling polymers, other more specific amounts can be useful or preferred for certain embodiments of dosage forms that include specific types of gelling polymer. For example, a dosage form that includes a carbomer polymer as a gelling polymer may include amounts across these broad ranges. Yet amounts of carbomer gelling polymer in a range from 2.5 to 35, or from 3 to 35, or from 5 to 35 weight percent carbomer gelling polymer based on a total weight of dosage form may be preferred as exhibiting especially desirable extraction resistance. As another example, a dosage form that includes xanthan gum as a gelling polymer may include amounts across the same above-recited broad ranges. Yet amounts of xanthan gum gelling polymer in a range from 1 to 20, e.g., 2 to 15, or from 3 to 12 weight percent xanthan gum gelling polymer, based on a total weight of dosage form, may be preferred as exhibiting especially desirable uptake resistance.

A useful gelling polymer can be any polymeric material that exhibits the ability to retain a significant fraction of adsorbed solvent in its molecular structure, e.g., the solvent being a solvent otherwise useful by an abuser to extract API from a dosage form or a crushed or powdered dosage form, the solvent for example being a commonly available solvent such as water or a C₁ to C₄ alcohol such as ethanol or methanol, etc. Examples of gelling polymers include materials that can swell or expand to a very high degree when placed in contact with such a solvent. The swelling or expansion may cause the gelling polymer to experience from a two- to one-thousand-fold volume increase from a dry state. Certain more specific examples of types of gelling polymers include swellable polymers sometimes referred to as osmopolymers or hydrogels. The gelling polymer may be non-crosslinked, lightly crosslinked, or highly crosslinked. The crosslinking may involve covalent or ionic bonds with the polymer possessing the ability to swell in the presence of a solvent, and when cross-linked will not dissolve in the solvent.

A gelling polymer, upon dissolution or dispersion in an aqueous solution or dispersion (e.g., water) at a concentration of 2% w/w (based on the dry material), creates a solution/dispersion with a viscosity of from about 100 to about 200,000 mPa·s (e.g., 4,000 to 175,000 mPa·s, and 4,000 to 50,000 mPa·s) as measured at 20 degrees Celsius (+/−0.2 degree Celsius) using the analysis method described in the USP 33 monograph for hypromellose (incorporated herein by reference).

Generally suitable gelling polymers include pharmaceutically acceptable polymers that undergo an increase in viscosity upon contact with a solvent, as described. Various examples of polymers are known to be useful in this manner, generally including natural and synthetic starches, natural and synthetic celluloses, acrylates, and polyalkylene oxides. Examples include polyethylene oxide, polyvinyl alcohol, hydroxypropyl methyl cellulose, methyl cellulose, hydroxyethylmethylcellulose, sodium carboxymethylcellulose, hydroxyethylcellulose, polyacrylic acid and polyvinyl carboxy polymers such as those commercially available under the trade name Carbopol®, and other high molecular weight polymers capable of attaining a viscosity level effective to prevent uptake in a syringe, if combined with a small volume of solvent as described.

Other examples of suitable gelling polymers can include: ethylcellulose, cellulose acetate, cellulose acetate propionate, cellulose acetate butyrate, cellulose acetate phthalate and cellulose triacetate, cellulose ether, cellulose ester, cellulose ester ether, cellulose; acrylic resins comprising copolymers synthesized from acrylic and methacrylic acid esters, for example acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

Exemplary gelling polymers can include natural polymers such as those derived from a plant or animal, as well as polymers prepared synthetically. Examples include polyhydroalkylcellulose having a molecular weight greater than 50,000; poly(hydroxyalkylmethacrylate) having a molecular weight of from 5,000 to 5,000,000; poly(vinylpyrrolidone) having a molecular weight of from 100,000 to 3,000,000; anionic and cationic hydrogels; poly(electrolyte) complexes; poly(vinylalcohol) having a low acetate residual; a swellable mixture of agar and carboxymethyl cellulose; a swellable composition comprising methyl cellulose mixed with a sparingly cross-linked agar; a polyether having a molecular weight of from 10,000 to 6,000,000; water-swellable copolymer produced by a dispersion of finely divided copolymer of maleic anhydride with styrene, ethylene, propylene, or isobutylene; water swellable polymer of N-vinyl lactams; and the like.

Other polymers useful as a gelling polymer include pectin having a molecular weight ranging from 30,000 to 300,000; polysaccharides such as agar, acacia, karaya, tragacanth, algins and guar; polyacrylamides; water-swellable indene maleic anhydride polymers; Good-Rite® polyacrylic acid having a molecular weight of 80,000 to 200,000; Polyox® polyethylene oxide polymers having a molecular weight of 100,000 to 7,000,000; starch graft copolymers; Aqua-Keep® acrylate polymers with water absorbability of 400 times its original weight; diesters of polyglucan; a mixture of cross-linked polyvinyl alcohol and poly(-vinyl-2-pyrrolidone); poly(ethylene glycol) having a molecular weight of 4,000 to 100,000.

In various specific embodiments, a gelling polymer may be or may include hydroxypropyl methyl cellulose (e.g., Hypromellose), and hydroxymethyl cellulose. The hydroxypropyl methyl cellulose can have a molecular weight ranging from 10,000 to 1,500,000. Examples of suitable, commercially available hydroxypropyl methylcellulose polymers include Methocel K100LV and Methocel K4M, available from Dow chemicals.

A specific class of preferred gelling polymer is the class of carbomer polymers, which are polymers derived from acrylic acid (e.g., acrylic acid homopolymers) and crosslinked with polyalcohol allyl ethers, e.g., crosslinked with polyalkenyl ethers of pentaerythritol or sucrose. Carbomer polymers have been shown to provide particularly useful extraction resistance in a dosage form, and therefore may be preferred in dosage forms as described, especially if present in an amount of at least 2.5, 3, or 5 weight percent based on a total weight of a dosage form.

Carbomer polymers are hydrophilic and are not substantially soluble in water. Rather, these polymers swell when dispersed in water forming a colloidal, mucilage-like dispersion. Carboxyl groups provided by acrylic acid residues of the polymer backbone are responsible for certain behavior of the polymers. Particles of this polymer can be viewed as a network structure of polymer chains interconnected by crosslinks. The structure can swell in water by up to one thousand times of an original (dry) volume (and ten times an original diameter of polymer particles) to form a gel when exposed to a pH environment above 4-6. The pKa of these polymers can be 6±0.5. Accordingly, carboxylate groups pendant from the polymer backbone can ionize at a pH above 6, producing a repulsion between the negatively-charged particles, which adds to the swelling of the polymer if exposed to solvent at this pH range. For this reason, a dosage form as described herein can preferably include a pH adjuster in an amount and location within the dosage form to raise the pH of a carbomer polymer to at least 6, to substantially neutralize the carboxylate groups.

Certain carbopol polymers that may be useful as a gelling polymer can have an average equivalent weight of 76 per carboxyl group. Examples of suitable commercially available carbomers include Carbopol® 934, 934P NF, Carbopol® 974P NF and Carbopol® 971P NF, Carbopol® 940, and Carbopol® 941, Carbopol® 71G, commercially available from Lubrizol. Examples of such polymers are described in U.S. Pat. Nos. 2,798,053 and 2,909,462, the entireties of which are incorporated herein by reference. Theoretical molecular weight ranges of Carbopol® products are in a range from 700,000 to 3 billion, theoretical estimation. For dosage forms as described herein, a gelling polymer (e.g., Carbopol®) can have a molecular weight and viscosity-increasing performance that will reduce or substantially inhibit an ability of an abuser to extract API from a combination of dosage form and a small volume of solvent, as described, while also being capable of being processed into a compresses dosage form.

A gelling polymer can also be characterized by viscosity of a solution prepared from the gelling polymer. Product information for commercially available Carbopol® polymers reports that viscosities of different Carbopol® polymers have the following viscosities:

| | Viscosity |
|---|---|
| Type of Carbomer | specified (cP) |
| Carbomer Homopolymer Type A (compendial name for | 4,000-11,000 |
| Carbopol 71G, Carbopol 971P and Carbopol 981) | |
| Carbomer Homopolymer Type B (compendial name for | 25,000-45,000 |
| Carbopol 934P, and Carbopol 934) | |
| Carbomer Homopolymer Type C (compendial name for | 40,000-60,000 |
| Carbopol 980) | |

(Type A and Type B viscosities measured using a Brookfield RVT, 20 rpm, neutralized to pH 7.3-7.8, 0.5 weight percent mucilage, spindle #5.)

Another example of a type of preferred gelling polymer is the class of xanthan gum polymers, which includes natural polymers useful as hydrocolloids, and derived from fermentation of a carbohydrate. A molecular weight of a Xanthan gum may be approximately 1,000,000. Xanthan gum has been shown to provide particularly useful extraction resistance in a dosage form as described, and therefore may be preferred in dosage forms as described, especially if present in an amount of at least 2 or 3 weight percent based on a total weight of a dosage form.

Without limiting the scope of useful gelling polymers to any specific type or molecular weight, examples of useful gelling polymers and useful respective molecular weights include the following:

| Gelling Polymer | Weight Average Molecular Weight |
|---|---|
| Carbomer | 700,000 to 3 billion (estimated) |
| HPMC 2910 K types | 164,000-1,200,000 |
| HPMC 2910 E types | 20,000-746,000 |
| Hydroxyethylcellulose | 90,000-1,300,000 |
| Ethylcellulose | 75,000-215,000 |
| Carboxymethylcellulose | 49,000-725,000 |
| Sodium Carboxymethylcellulose | 49,000-725,000 |
| Povidone | 4,000-1,300,000 |
| Copovidone | 47,000 |
| Hydroxypropyl cellulose | 40,000-1,150,000 |
| Xanthan Gum | 1,000,000 |
| Polyethylene oxide | Average molecular wt: |
| | 100,000-7,000,000 |

The dosage form may optionally include filler, which may be present in the dosage form at a location and in an amount to also not interfere with desired uptake of the active pharmaceutical ingredient by a patient upon oral ingestion in an immediate release dosage form. An exemplary location for the filler is as a component of a compressed matrix of a dosage form such as a compressed tablet or a compressed capsule. When a filler is mixed with the active pharmaceutical ingredient, such as by mechanically grinding a dosage form, the active pharmaceutical ingredient is inhibited or prevented from becoming thereafter dissolved in a solvent such as water or otherwise efficiently accessed by an abuser.

Examples of fillers that may be useful in an immediate release dosage form as described include lactose, starch, dextrose, sucrose, fructose, maltose, mannitol, sorbitol, kaolin, microcrystalline cellulose, powdered cellulose, calcium sulfate, calcium phosphate, dicalcium phosphate, lactitol, or any combination of two or more of these. As compared to non-filler ingredients such as gelling polymers, a filler is of a molecular weight that does not result in a substantial viscosity increase or formation of a gel as described herein for a gelling polymer, if combined with a solvent such as water.

The filler may be present at any one or more of these portions of a dosage form in an amount to provide desired processing or functional properties of a portion of the dosage form and of the entire dosage form. The amount of total filler in a dosage form can also be as desired to provide desired functionality, including an immediate release profile, for example in an amount in a range from 0 to 80 weight percent filler based upon the total weight of the dosage form, e.g., from 3 to 80 or from 5 to 50 percent filler based on total weight dosage form.

Certain preferred dosage forms as described include a combination of fillers that includes mannitol and microcrystalline cellulose. According to these dosage forms, the amounts and relative amounts of the mannitol and microcrystalline cellulose can be any that are desired and useful to provide an immediate release dosage form as described, especially having useful or advantageous abuse deterrent features. For example, mannitol may be present in such a dosage form in a range from 1 to 60 weight percent, e.g., from 1 to 15 weight percent, based on a total weight of the dosage form. Microcrystalline cellulose may be present in such a dosage from in a range from 6 to 80 weight percent, e.g., in a range from 15 to 50 weight percent based on a total weight of the dosage form.

A dosage form as presently described can also preferably include a disintegrant, which functions to cause the dosage form to expand and break up during use, e.g., at conditions of a human stomach, to allow active pharmaceutical ingredient of the dosage form to be released in a manner to achieve an immediate release profile. Disintegrants are known ingredients of pharmaceutical dosage forms, with various examples being known and commercially available. Examples of disintegrants include compositions of or containing sodium starch glycolate, starch (e.g., Maize starch, Potato starch, Rice Starch, Tapioca Starch, Wheat Starch, Corn Starch and pregelatinized starch), croscarmellose sodium, crospovidone (crosslinked polyvinyl N-pyrrolidone or PVP) (polyplasdone XL-10), sodium starch glycolate (EXPLOTAB® or PRIMOJEL®), any combination of two or more of the foregoing, and other pharmaceutically acceptable materials formed into particles having a having particle size, density, etc., to allow processing of the disintegrant into a useful immediate release dosage form.

The disintegrant can be present in an immediate release dosage form at any location that allows the disintegrant to function as desired, to expand within the intact dosage form, upon ingestion, to cause the ingested dosage form to break apart and allow for desired immediate release of active pharmaceutical ingredient from the dosage form, in a stomach. An exemplary location for the gelling polymer is as a component of a compressed matrix of a dosage form such as a compressed tablet or a compressed capsule.

When included in a compressed matrix of a dosage form, disintegrant may be present in an amount useful to achieve immediate release of an API of a dosage form. Examples of useful amounts of disintegrant in an immediate release dosage form as described herein may be in a range from 10 to 40 weight percent disintegrant based on a total weight of the dosage form, e.g., from 12 to 35 weight percent disintegrant based on total weight dosage form.

A dosage form as described can also include any of various known and conventional pharmaceutical excipients that may be useful to achieve desired processing and performance properties of an immediate release dosage form. These excipients include binders, lubricants, glidants, coloring agents, pH-adjusters, etc. A more detailed description of pharmaceutical excipients that may also be included in the tablets of the present invention can be found in The Handbook of Pharmaceutical Excipients, 5th ed. (2006).

Examples of binders that may be included in a dosage form as described include polymeric material such as alginic acid, sodium carboxymethylcellulose, microcrystalline cellulose, dextrin, ethylcellulose, gelatin, starch, pregelatinized starch, polyvinyl alcohol, polyethylene oxide, polyvinylpyrrolidone, polyacrylamides, polyvinyloxoazolidone, polyvinylalcohols, methylcellulose, hydroxypropyl cellulose, hydroxymethyl cellulose and any combination of two or more of these. A binder may be a water soluble material; as compared to non-binder ingredients such as a gelling polymer, a binder is of a molecular weight that does not result in formation of a gel or a highly viscous (sufficient to prevent abuse) composition upon combining with a small volume of water. A binder can exhibit a relatively low molecular weight as compared to a gelling polymer, and a relatively lower viscosity (e.g., when measured in a 2% aqueous solution). Polymer useful as a binder may typically have a molecular weight of less than 50,000, e.g., less than 30,000, or than 10,000.

The amount of total binder in a dosage form can also be as desired to provide desired functionality, including immediate release functionality, for example in an amount in a range from 0.1 to 10 weight percent binder based on a total weight of a dosage form, e.g., from 0.5 to 7 weight percent binder based on total weight dosage form.

A pH-adjuster can be included in an immediate release dosage form as described, for example at a location to affect pH at a specific location of the dosage form that is only a portion of a total dosage form. As an example, a pH-adjuster in the form of a base may be included at a location of a gelling polymer useful to adjust pH of a dosage form that contains an acidic component. The acidic component may be any component, such as an acidic polymer, e.g., a carbomer gelling polymer. An example of a useful basic pH-adjuster is sodium bicarbonate, but other known basic and acidic pharmaceutically acceptable pH-adjusters are known and commercially available.

An exemplary location for the gelling polymer is as a component of a compressed matrix of a dosage form such as a compressed tablet or a compressed capsule. When included in a compressed matrix of a dosage form, pH-adjuster may be present in an amount useful to achieve a desired pH of a dosage form in combination with immediate release of an API of a dosage form and, preferably, desired abuse deterrent features. Examples of useful amounts of pH-adjuster (e.g., sodium bicarbonate) in an immediate release dosage form as described may be in a range from 0.5 to 50 weight percent pH-adjuster based on a total weight of the dosage form, e.g., from 1 to 8 weight percent pH-adjuster based on total weight dosage form.

Examples of lubricants include talc, glyceryl monostearates, calcium stearate, magnesium stearate, stearic acid, glyceryl behenate, polyethylene glycol, poloxamer and combinations of the foregoing. Lubricant may be included in an immediate release dosage form as described, in any useful amount, such as an amount in a range from 0.1 to 10 weight percent lubricant based on a total weight of a dosage form, e.g., from 0.5 to 7 weight percent lubricant based on total weight dosage form.

Examples of glidants include colloidal silicon dioxide, untreated fumed silica (e.g., as available under the trade name Cab-O-Sil®), and crystalline or fused quartz. Glidant may be included in an immediate release dosage form as described, in any useful amount.

Examples of coloring agents include FD&C-type dyes and lakes, fruit and vegetable extracts, titanium dioxide and mixtures thereof. A coloring agent may be incorporated into a dosage form by blending the coloring agent any other ingredient. Alternately, coloring agent may be applied to an outer surface of a dosage form.

Any active pharmaceutical ingredient alone or in combination can be included in an immediate release dosage form as described herein. With abuse deterrent features as described herein, some being operative based on specific structural or compositional features of the dosage form, APIs that can be particularly useful can be those types of active pharmaceutical ingredients that can be subject to abuse, addiction, overdosing, or two or more of these. Such APIs can be located in the dosage form at a location to cause the API to be subject to the abuse deterrent features.

Drugs commonly susceptible to abuse include sedative-hypnotics, stimulants, anxiolytics, and narcotic analgesics including but not limited to drugs that can cause psychological or physical dependence on the drug. An API can include any therapeutically acceptable drug salt, drug derivative, drug analog, drug homologue, or polymorph of an active pharmaceutical ingredient.

Sedative hypnotics include, for example, barbiturates, for example phenobarbital, methobarbital, amobarbital, pentobarbital, and secobarbital and pharmaceutically acceptable salts thereof; benzodiazepines, for example diazepam, chlorodiazepoxide, lorazepam, triazolam, temazepam, alprazolam and flurazepam and pharmaceutically acceptable salts thereof; phenothiazines, such as for example, alimemazine, chlorpromazine, thioridazine, and pharmaceutically acceptable salts thereof, and sleep medications, such as for example, zolpidem, zaleplon, and eszopiclone and pharmaceutically acceptable salts thereof. Anxiolytics include, for example, benzodiazepines, for example diazepam, chlordiazepoxide, estazolam, lorazepam, triazolam, alprazolam, clonazepam and flurazepam and pharmaceutically acceptable salts thereof. Stimulants include, for example, pseudoephedrine, amphetamines, such as for example, dextroamphetamine, levoamphetamine (benzadrine), methamphetamine (methadrine), pseudoephedrine, and Adderall (amphetamine mixed salts) and pharmaceutically acceptable salts thereof, and non-amphetamine psychostimulants such as methylphenidate, modafinil and armodafinil and pharmaceutically acceptable salts thereof. Narcotic analgesics include opioids such as, for example, buprenorphine, butorphanol, codeine, dihydrocodeine, dihydromorphine, hydrocodone, hydromorphone, morphine, oxycodone, oxymorphone, methadone, fentanyl, meperidine, tramadol, propoxyphene, and pharmaceutically acceptable salts thereof.

Other specific drugs which may be susceptible to abuse include for example, muscle relaxants such as for example cyclobenzaprine and pharmaceutically acceptable salts thereof, cannabinoids, such as dronabinol and pharmaceutically acceptable salts thereof.

The amount of active pharmaceutical ingredient included in an immediate release dosage form can be any useful amount, as is known and as may be found in relevant literature such as Goodman & Gillman's, The Pharmacological Basis of Therapeutics, 9th ed. pages 219-222, 361-396, 521-535 1996. For example, typical therapeutic amounts of oxycodone range 5 mg, 10 mg, or up to 400 mg, for the hydrochloride salt. Often, when processed into a suitable immediate release dosage form, the active pharmaceutical ingredient can be present in such dosage form in an amount normally prescribed, typically 0.5 to 25 percent on a dry weight basis, based on the total weight of the dosage form. With respect to narcotic analgesics such as opioids in a single unit dosage form, such as at a level from about 1 to about 500 mg, or from about 1 to about 250 mg, or from about 1 to about 100 mg; for example, 2.5, 5, 7.5, 10, 15, 20, or 30, milligram (mg) per dosage form unit. In other embodiments, a dosage form contains any appropriate amount of an API to provide a therapeutic effect.

A dosage form as described can optionally include one or more additional APIs of a type that is not commonly susceptible to abuse. This additional API may be any suitable or desired API, such as those in the class of non-steroidal analgesic drugs. The expression “non-steroidal analgesic drugs” as used herein refers to drugs that include those commonly referred to as non-steroidal anti-inflammatory drugs, or “NSAIDS,” and acetaminophen, which is non-steroidal, but does not act via an inflammation mechanism. Accordingly, the term “non-steroidal analgesic drugs” would include acetaminophen, and also include NSAIDS such as aspirin, ibuprofen, and naproxen. These APIs being not commonly susceptible to abuse, they need not be located in the dosage form at a location at which an abuse deterrent feature would protect the API from potential abuse. The dosage form also exhibits immediate release properties with respect to these not-commonly-subject-to-abuse APIs. And these APIs can be present in the dosage form at any useful level, typically 0.5 to 25, e.g., 1 to 10 weight percent of the API on a dry weight basis, based on a total weight of the dosage form, e.g., at a level of or between any of 5, 25, 50, 60, 75, 100, 125, 150, 175, 200, 300, 325, 500, 750 or up to or exceeding 1000 milligram (mg) per dosage form unit. In other embodiments, a dosage form contains an appropriate amount of an API to provide a therapeutic effect.

An immediate release dosage form as described can include an abuse deterrent feature as described, e.g., gelling polymer, along with a filler and disintegrant. With the described abuse deterrent feature, other types of known abuse deterrent features may not be necessary and may be specifically excluded from an immediate release dosage form as described. Certain embodiments of the described dosage forms can specifically exclude other types of abuse deterrents.

In specific, some dosage forms include nasal irritant to discourage or prevent abuse by nasal insufflation. The nasal irritant can be a mucous membrane irritant or nasal passageway irritant that, if inhaled through a nasal passageway when contained in a ground or powdered dosage form, can induce pain or irritation of the abuser's nasal passageway tissue. Examples include surfactants such as sodium lauryl sulfate, poloxamer, sorbitan monoesters, and glyceryl monooleates. Certain particular embodiments of dosage forms of the present description do not require, and can specifically exclude, nasal irritant agents such as those described above

Alternately, dosage forms can include an emetic agent, to cause vomiting. Certain particular embodiments of dosage forms of the present description do not require, and can specifically exclude, an emetic agent.

Alternately, some dosage forms include an effervescent agent that acts as a deterrent to abuse by nasal insufflation. The effervescent includes an acidic component and a basic component that release a gas such as oxygen or carbon dioxide when combined in the presence of an aqueous media, such as upon nasal insufflation. See, e.g., patent publication WO 2013/077851, the entirety of which is incorporated herein by reference. The acid source may be, for example, citric acid, tartaric acid, malic acid, maleic acid, lactic acid, glycolic acid, ascorbic acid, fumaric acid, adipic acid, succinic acid, salts thereof, and combinations thereof. The base may be, for example, a carbonate or bicarbonate. Dosage forms of the present description do not require, and can specifically exclude, an effervescent in the form of an acid and a base that can combine to a gas such as oxygen or carbon dioxide.

Still other dosage forms include a biologically active chemical compound that functions as an antagonist to an active pharmaceutical ingredient. An antagonist may prevent the potential abuse of a dosage form in a manner, including the method of consuming multiple or several or more dosage form units at once. Antagonist agents are compounds that block or negate the effect of an active pharmaceutical ingredient, and are available and known for various classes of drugs including opioids and other pharmaceutical agents. Examples of antagonist agents for opioids include compounds such as naltrexone, naloxone, nalmefene, cyclazacine, levallorphan. Specific examples of antagonist agents and methods for preparing antagonist agents for incorporation into a dosage formare provided in U.S. Pat. Nos. 7,682,633 and 7,658,939, which are incorporated herein by reference. According to the present description, an immediate release dosage form that includes an opioid and that includes one or more abuse deterrent feature as described herein (e.g., a gelling polymer, wax, solvent-resistant film, or a combination thereof), can be formulated to not contain and to specifically exclude an agonist of an API that is also included in the dosage form, e.g., an opioid antagonist in a dosage form containing an opioid.

The following non-limiting examples show various dosage forms as described herein. The described and exemplified dosage forms can be made from methods that include mixing and compressing steps as follows.

Paracetamol quantity is adjusted for assay of acetaminophen. The adjustment is made by varying the amount of mannitol.

Hydrocodone bitartrate, Paracetamol DC272N, crospovidone, gelling polymer, sodium bicarbonate (if needed) and mannitol and MCC along with any other excipients as indicated in tables below are blended.

Magnesium stearate is then added to the blender and mixed.

The blend is compressed into capsule shaped tablets using a rotary tablet press.

Tablet friability is preferably less than 1.0%.

### Example 1

| Ingredient | % w/w | mg/tab |
|---|---|---|
| Hydrocodone Bitartrate | 0.50% | 5.00 |
| APAP (96.4%) | 33.71% | 337.10 |
| Mannitol | 12.39% | 123.90 |
| Carbopol | 1.50% | 15.00 |
| MCC | 20.00% | 200.00 |
| Crospovidone | 30.00% | 300.00 |
| Sodium Bicarbonate | 0.90% | 9.00 |
| Magnesium Stearate | 1.00% | 10.00 |
| Total | 100.00% | 1000.00 |

### Example 2

| Ingredient | % w/w | mg/tab |
|---|---|---|
| Hydrocodone Bitartrate | 0.50% | 5.00 |
| APAP (96.4%) | 33.71% | 337.10 |
| Mannitol EZ | 9.99% | 99.90 |
| Carbopol | 3.00% | 30.00 |
| MCC | 20.00% | 200.00 |
| Crospovidone | 30.00% | 300.00 |
| Sodium Bicarbonate | 1.80% | 18.00 |
| Magnesium Stearate | 1.00% | 10.00 |
| Total | 100.00% | 1000.00 |

### Example 3

| Ingredient | % w/w | mg/tab |
|---|---|---|
| Hydrocodone Bitartrate | 0.50% | 5.00 |
| APAP (96.4%) | 33.71% | 337.10 |
| Mannitol | 6.79% | 67.90 |
| Carbopol | 5.00% | 50.00 |
| MCC | 20.00% | 200.00 |
| Crospovidone | 30.00% | 300.00 |
| Sodium Bicarbonate | 3.00% | 30.00 |
| Magnesium Stearate | 1.00% | 10.00 |
| Total | 100.00% | 1000.00 |

### Example 4

| Ingredient | % w/w | mg/tab |
|---|---|---|
| Hydrocodone Bitartrate | 0.50% | 5.00 |
| APAP (96.4%) | 33.71% | 337.10 |
| Mannitol EZ | 9.99% | 99.90 |
| Carbopol | 3.00% | 30.00 |
| Crospovidone | 30.00% | 300.00 |
| Sodium Bicarbonate | 1.80% | 18.00 |
| Magnesium Stearate | 1.00% | 10.00 |
| Total | 80.00% | 800.00 |

### Example 5

| Ingredient | % w/w | mg/tab |
|---|---|---|
| Hydrocodone Bitartrate | 0.50% | 5.00 |
| APAP (96.4%) | 33.71% | 337.10 |
| Mannitol | 9.79% | 97.90 |
| Xanthan Gum | 5.00% | 50.00 |
| MCC | 20.00% | 200.00 |
| Crospovidone | 30.00% | 300.00 |
| Magnesium Stearate | 1.00% | 10.00 |
| Total | 100.00% | 1000.00 |

### Example 6

| Ingredient | % w/w | mg/tab |
|---|---|---|
| Hydrocodone Bitartrate | 1.00% | 10.00 |
| APAP (96.4%) | 33.71% | 337.10 |
| Mannitol | 8.29% | 82.90 |
| Carbopol | 10.00% | 100.00 |
| MCC | 15.00% | 150.00 |
| Crospovidone | 25.00% | 250.00 |
| Sodium Bicarbonate | 6.00% | 60.00 |
| Magnesium Stearate | 1.00% | 10.00 |
| Total | 100.00% | 1000.00 |

### Example 7

| Ingredient | % w/w | mg/tab |
|---|---|---|
| Hydrocodone Bitartrate | 1.25% | 10.00 |
| APAP (96.4%) | 42.14% | 337.14 |
| Mannitol | 2.61% | 20.86 |
| Carbopol | 12.50% | 100.00 |
| MCC | 13.00% | 104.00 |
| Crospovidone | 20.00% | 160.00 |
| Sodium Bicarbonate | 7.50% | 60.00 |
| Magnesium Stearate | 1.00% | 8.00 |
| Total | 100.00% | 800.00 |

### Example 8

| Ingredient | % w/w | mg/tab |
|---|---|---|
| Oxycodone HCl* | 0.49% | 5.15 |
| APAP (96.8%) | 31.98% | 335.79 |
| Mannitol | 11.53% | 121.07 |
| HPMC K100M | 5.00% | 52.50 |
| MCC | 20.00% | 210.00 |
| Crospovidone | 30.00% | 315.00 |
| Magnesium Stearate | 1.00% | 10.50 |
| Total | 100.00% | 1050.00 |

*adjusted for moisture content of 2.8%

### Example 9

| Ingredient | % w/w | mg/tab |
|---|---|---|
| Oxycodone HCl* | 0.49% | 5.15 |
| APAP (96.8%) | 31.98% | 335.79 |
| Mannitol | 9.03% | 94.82 |
| HPMC K100M | 7.50% | 78.75 |
| MCC | 20.00% | 210.00 |
| Crospovidone | 30.00% | 315.00 |
| Magnesium Stearate | 1.00% | 10.50 |
| Total | 100.00% | 1050.00 |

*adjusted for moisture content of 2.8%

### Example 10

| Ingredient | % w/w | mg/tab |
|---|---|---|
| Oxycodone HCl* | 0.49% | 5.15 |
| APAP (96.8%) | 31.98% | 335.79 |
| Mannitol | 9.03% | 94.82 |
| HPMC K100 LV | 7.50% | 78.75 |
| MCC | 20.00% | 210.00 |
| Crospovidone | 30.00% | 315.00 |
| Magnesium Stearate | 1.00% | 10.50 |
| Total | 100.00% | 1050.00 |

*adjusted for moisture content of 2.8%

### Example 11

| Ingredient | % w/w | mg/tab |
|---|---|---|
| Oxycodone HCl* | 0.49% | 5.15 |
| APAP (96.8%) | 31.98% | 335.79 |
| Mannitol | 1.53% | 16.07 |
| HPMC K100 LV | 15.00% | 157.50 |
| MCC | 20.00% | 210.00 |
| Crospovidone | 30.00% | 315.00 |
| Magnesium Stearate | 1.00% | 10.50 |
| Total | 100.00% | 1050.00 |

*adjusted for moisture content of 2.8%

### Example 12

| Ingredient | % w/w | mg/tab |
|---|---|---|
| Oxycodone HCl* | 0.49% | 5.15 |
| APAP (96.8%) | 31.98% | 335.79 |
| Mannitol | 13.03% | 136.82 |
| PEO (2 MM MW) | 3.50% | 36.75 |
| MCC | 20.00% | 210.00 |
| Crospovidone | 30.00% | 315.00 |
| Magnesium Stearate | 1.00% | 10.50 |
| Total | 100.00% | 1050.00 |

*adjusted tor moisture content of 2.8%

### Example 13

| Ingredient | % w/w | mg/tab |
|---|---|---|
| Oxycodone HCl* | 0.49% | 5.15 |
| APAP (96.8%) | 31.98% | 335.79 |
| Mannitol | 11.53% | 121.07 |
| PEO (2 MM MW) | 5.00% | 52.50 |
| MCC | 20.00% | 210.00 |
| Crospovidone | 30.00% | 315.00 |
| Magnesium Stearate | 1.00% | 10.50 |
| Total | 100.00% | 1050.00 |

*adjusted for moisture content of 2.8%

### APAP Evaluation

### Example 14

| Ingredient | % w/w | mg/tab |
|---|---|---|
| APAP (96.8%) | 31.98% | 335.79 |
| Mannitol | 9.52% | 99.96 |
| HPMC K100M | 7.50% | 78.75 |
| MCC | 20.00% | 210.00 |
| Crospovidone | 30.00% | 315.00 |
| Magnesium Stearate | 1.00% | 10.50 |
| Total | 100.00% | 1050.00 |

### APAP Evaluation

### Example 15

| Ingredient | % w/w | mg/tab |
|---|---|---|
| APAP (97.4%) | 31.78% | 333.69 |
| Mannitol | 9.72% | 102.06 |
| HPMC K100M | 7.50% | 78.75 |
| MCC | 20.00% | 210.00 |
| Crospovidone | 30.00% | 315.00 |
| Magnesium Stearate | 1.00% | 10.50 |
| Total | 100.00% | 1050.00 |

### APAP Evaluation

### Example 16

| Ingredient | % w/w | mg/tab |
|---|---|---|
| APAP (94.8%) | 32.65% | 342.83 |
| Mannitol | 8.85% | 92.93 |
| HPMC K100M | 7.50% | 78.75 |
| MCC | 20.00% | 210.00 |
| Crospovidone | 30.00% | 315.00 |
| Magnesium Stearate | 1.00% | 10.50 |
| Total | 100.00% | 1050.00 |

### Example 17

| Ingredient | % w/w | mg/tab |
|---|---|---|
| APAP (94.8%) | 48.97% | 342.79 |
| Mannitol | 15.03% | 105.21 |
| PEO | 5.00% | 35.00 |
| MCC | 20.00% | 140.00 |
| Crospovidone | 10.00% | 70.00 |
| Magnesium Stearate | 1.00% | 7.00 |
| Total | 100.00% | 700.00 |

### Example 18

| Ingredient | % w/w | mg/tab |
|---|---|---|
| APAP (94.8%) | 34.28% | 342.80 |
| Mannitol | 9.72% | 97.20 |
| PEO | 5.00% | 50.00 |
| MCC | 20.00% | 200.00 |
| Crospovidone | 30.00% | 300.00 |
| Magnesium Stearate | 1.00% | 10.00 |
| Total | 100.00% | 1000.00 |

### Example 19

| Ingredient | % w/w | mg/tab |
|---|---|---|
| APAP (94.8%) | 34.28% | 342.80 |
| Mannitol | 11.72% | 117.20 |
| Xanthan Gum | 3.00% | 30.00 |
| MCC | 20.00% | 200.00 |
| Crospovidone | 30.00% | 300.00 |
| Magnesium Stearate | 1.00% | 10.00 |
| Total | 100.00% | 1000.00 |

### Example 20

| Ingredient | % w/w | mg/tab |
|---|---|---|
| APAP (94.8%) | 34.28% | 342.80 |
| Mannitol | 13.22% | 132.20 |
| Xanthan Gum | 1.50% | 15.00 |
| MCC | 20.00% | 200.00 |
| Crospovidone | 30.00% | 300.00 |
| Magnesium Stearate | 1.00% | 10.00 |
| Total | 100.00% | 1000.00 |

### Example 21

| Ingredient | % w/w | mg/tab |
|---|---|---|
| APAP (94.8%) | 34.28% | 342.80 |
| Mannitol | 4.72% | 47.20 |
| Kollidon 90F | 10.00% | 100.00 |
| MCC | 20.00% | 200.00 |
| Crospovidone | 30.00% | 300.00 |
| Magnesium Stearate | 1.00% | 10.00 |
| Total | 100.00% | 1000.00 |

### Example 22

| Ingredient | % w/w | mg/tab |
|---|---|---|
| Hydrocodone Bitartrate | 0.50% | 5.00 |
| APAP (94.8%) | 34.28% | 342.80 |
| Mannitol | 9.22% | 92.20 |
| PEO | 5.00% | 50.00 |
| MCC | 20.00% | 200.00 |
| Crospovidone | 30.00% | 300.00 |
| Magnesium Stearate | 1.00% | 10.00 |
| Total | 100.00% | 1000.00 |

### Example 23

| Ingredient | % w/w | mg/tab |
|---|---|---|
| Hydrocodone Bitartrate | 0.50% | 5.00 |
| APAP (94.8%) | 34.28% | 342.80 |
| Mannitol | 13.22% | 132.20 |
| Xanthan Gum | 1.00% | 10.00 |
| MCC | 20.00% | 200.00 |
| Crospovidone | 30.00% | 300.00 |
| Magnesium Stearate | 1.00% | 10.00 |
| Total | 100.00% | 1000.00 |

### Example 24

| Ingredient | % w/w | mg/tab |
|---|---|---|
| Hydrocodone Bitartrate | 0.50% | 5.00 |
| APAP (96.4%) | 33.71% | 337.10 |
| Mannitol | 4.79% | 47.90 |
| Kollidon 90F | 20.00% | 200.00 |
| MCC | 15.00% | 150.00 |
| Crospovidone | 25.00% | 250.00 |
| Magnesium Stearate | 1.00% | 10.00 |
| Total | 100.00% | 1000.00 |

### Example 25

| Ingredient | % w/w | mg/tab |
|---|---|---|
| Hydrocodone Bitartrate | 0.71% | 4.97 |
| APAP (96.4%) | 48.16% | 337.12 |
| Mannitol | 10.13% | 70.91 |
| PEO | 5.00% | 35.00 |
| MCC | 20.00% | 140.00 |
| Crospovidone | 15.00% | 105.00 |
| Magnesium Stearate | 1.00% | 7.00 |
| Total | 100.00% | 700.00 |

### Example 26

| Ingredient | % w/w | mg/tab |
|---|---|---|
| Hydrocodone Bitartrate | 0.50% | 5.00 |
| APAP (96.4%) | 33.71% | 337.10 |
| Mannitol | 11.79% | 117.90 |
| Xanthan Gum | 3.00% | 30.00 |
| MCC | 20.00% | 200.00 |
| Crospovidone | 30.00% | 300.00 |
| Magnesium Stearate | 1.00% | 10.00 |
| Total | 100.00% | 1000.00 |

### Example 27

| Ingredient | % w/w | mg/tab |
|---|---|---|
| Hydrocodone Bitartrate | 0.50% | 5.00 |
| APAP (96.4%) | 33.71% | 337.10 |
| Mannitol | 14.79% | 147.90 |
| Gum Arabic | 20.00% | 200.00 |
| MCC | 10.00% | 100.00 |
| Crospovidone | 20.00% | 200.00 |
| Magnesium Stearate | 1.00% | 10.00 |
| Total | 100.00% | 1000.00 |

### Example 28

| Ingredient | % w/w | mg/tab |
|---|---|---|
| Hydrocodone Bitartrate | 1.110% | 9.99 |
| APAP (96.4%) | 37.460% | 337.14 |
| Mannitol | 2.650% | 23.85 |
| Carbopol | 11.110% | 99.99 |
| MCC | 15.000% | 135.00 |
| Crospovidone | 25.000% | 225.00 |
| Sodium Bicarbonate | 6.670% | 60.03 |
| Magnesium Stearate | 1.000% | 9.00 |
| Total | 100.000% | 900.00 |

### Example 29

| Ingredient | % w/w | mg/tab |
|---|---|---|
| Hydrocodone Bitartrate | 1.429% | 10.00 |
| APAP (96.4%) | 48.162% | 337.13 |
| Mannitol | 2.266% | 15.86 |
| Carbopol | 14.286% | 100.00 |
| Crospovidone | 26.000% | 182.00 |
| Sodium Bicarbonate | 6.857% | 48.00 |
| Magnesium Stearate | 1.000% | 7.00 |
| Total | 100.000% | 700.00 |

### Example 30

| Ingredient | % w/w | mg/tab |
|---|---|---|
| Hydrocodone Bitartrate | 1.250% | 10.00 |
| APAP (96.4%) | 42.142% | 337.14 |
| Mannitol | 1.483% | 11.86 |
| Carbopol | 12.500% | 100.00 |
| MCC | 6.000% | 48.00 |
| Crospovidone | 28.125% | 225.00 |
| Sodium Bicarbonate | 7.500% | 60.00 |
| Magnesium Stearate | 1.000% | 8.00 |
| Total | 100.000% | 800.00 |

### Example 31

| Ingredient | % w/w | mg/tab |
|---|---|---|
| Hydrocodone Bitartrate | 1.250% | 10.00 |
| APAP (96.4%) | 42.142% | 337.14 |
| Mannitol | 1.038% | 8.30 |
| Carbopol | 12.500% | 100.00 |
| Crospovidone | 34.500% | 276.00 |
| Red Iron Oxide 212P | 0.070% | 0.56 |
| Sodium Bicarbonate | 7.500% | 60.00 |
| Magnesium Stearate | 1.000% | 8.00 |
| Total | 100.000% | 800.00 |

### Example 32

| Ingredient | % w/w | mg/tab |
|---|---|---|
| Hydrocodone Bitartrate | 1.111% | 10.00 |
| APAP (96.4%) | 37.460% | 337.14 |
| Mannitol | 2.591% | 23.32 |
| Carbopol | 11.111% | 100.00 |
| MCC | 15.000% | 135.00 |
| Crospovidone | 25.000% | 225.00 |
| Red Iron Oxide 212P | 0.06% | 60.00 |
| Sodium Bicarbonate | 6.667% | 0.54 |
| Magnesium Stearate | 1.000% | 9.00 |
| Total | 100.00% | 900.00 |

### Example 33

| Ingredient | % w/w | mg/tab |
|---|---|---|
| Hydrocodone Bitartrate | 1.111% | 10.00 |
| APAP (96.4%) | 37.460% | 337.14 |
| Mannitol | 2.651% | 23.86 |
| Carbopol | 11.111% | 100.00 |
| MCC | 15.000% | 135.00 |
| Crospovidone | 25.000% | 225.00 |
| Sodium Bicarbonate | 6.667% | 60.00 |
| Magnesium Stearate | 1.000% | 9.00 |
| Total | 100.00% | 900.00 |

### Example 34

| Ingredient | % w/w | mg/tab |
|---|---|---|
| APAP (95.1%) | 37.972% | 341.75 |
| Mannitol | 3.139% | 28.25 |
| Carbopol | 5.556% | 50.00 |
| MCC | 19.000% | 171.00 |
| Crospovidone | 30.000% | 270.00 |
| Sodium Bicarbonate | 3.333% | 30.00 |
| Magnesium Stearate | 1.000% | 9.00 |
| Total | 100.000% | 900.00 |

### Example 35

| Ingredient | % w/w | mg/tab |
|---|---|---|
| APAP (95.1%) | 37.972% | 341.75 |
| Mannitol EZ | 3.583% | 32.25 |
| Carbopol | 7.778% | 70.00 |
| MCC | 15.000% | 135.00 |
| Crospovidone | 30.000% | 270.00 |
| Sodium Bicarbonate | 4.667% | 42.00 |
| Magnesium Stearate | 1.000% | 9.00 |
| Total | 100.000% | 900.00 |

### Example 36

| Ingredient | % w/w | mg/tab |
|---|---|---|
| APAP (95.1%) | 37.972% | 341.75 |
| Mannitol | 3.250% | 29.25 |
| Carbopol | 11.111% | 100.00 |
| MCC | 15.000% | 135.00 |
| Crospovidone | 25.000% | 225.00 |
| Sodium Bicarbonate | 6.667% | 60.00 |
| Magnesium Stearate | 1.000% | 9.00 |
| Total | 100.000% | 900.00 |

### Example 37

| Ingredient | % w/w | mg/tab |
|---|---|---|
| APAP (95.1%) | 37.972% | 341.75 |
| Mannitol | 12.139% | 109.25 |
| Carbopol | 5.556% | 50.00 |
| MCC | 15.000% | 135.00 |
| Crospovidone | 25.000% | 225.00 |
| Sodium Bicarbonate | 3.333% | 30.00 |
| Magnesium Stearate | 1.000% | 9.00 |
| Total | 100.000% | 900.00 |

### Example 38

| Ingredient | % w/w | mg/tab |
|---|---|---|
| Hydrocodone Bitartrate | 1.110% | 9.99 |
| APAP (96.4%) | 37.460% | 337.14 |
| Mannitol | 14.870% | 133.83 |
| Xanthan Gum | 5.560% | 50.04 |
| MCC | 15.000% | 135.00 |
| Crospovidone | 25.000% | 225.00 |
| Magnesium Stearate | 1.000% | 9.00 |
| Total | 100.000% | 900.00 |

### Example 39

| Ingredient | % w/w | mg/tab |
|---|---|---|
| Hydrocodone Bitartrate | 1.250% | 10.00 |
| APAP (96.4%) | 42.140% | 337.12 |
| Mannitol | 9.360% | 74.88 |
| Xanthan Gum | 6.250% | 50.00 |
| MCC | 15.000% | 120.00 |
| Crospovidone | 25.000% | 200.00 |
| Magnesium Stearate | 1.000% | 8.00 |
| Total | 100.000% | 800.0 0 |

### Example 40

| Ingredient | % w/w | mg/tab |
|---|---|---|
| Hydrocodone Bitartrate | 1.250% | 10.00 |
| APAP (96.4%) | 42.140% | 337.12 |
| Mannitol | 9.610% | 76.88 |
| Carbopol | 3.750% | 30.00 |
| MCC | 15.000% | 120.00 |
| Crospovidone | 25.000% | 200.00 |
| Sodium Bicarbonate | 2.250% | 18.00 |
| Magnesium Stearate | 1.000% | 8.00 |
| Total | 100.000% | 800.00 |

### Example 41

| Ingredient | % w/w | mg/tab |
|---|---|---|
| Hydrocodone Bitartrate | 1.000% | 10.00 |
| APAP (96.4%) | 33.710% | 337.10 |
| Mannitol | 9.290% | 92.90 |
| Xanthan Gum | 5.000% | 50.00 |
| MCC | 20.000% | 200.00 |
| Crospovidone | 30.000% | 300.00 |
| Magnesium Stearate | 1.000% | 10.00 |
| Total | 100.000% | 1000.00 |

### Example 42

| Ingredient | % w/w | mg/tab |
|---|---|---|
| Hydrocodone Bitartrate | 1.250% | 10.00 |
| APAP (96.4%) | 42.140% | 337.12 |
| Mannitol | 5.610% | 44.88 |
| Carbopol | 6.250% | 50.00 |
| MCC | 15.000% | 120.00 |
| Crospovidone | 25.000% | 200.00 |
| Sodium Bicarbonate | 3.750% | 30.00 |
| Magnesium Stearate | 1.000% | 8.00 |
| Total | 100.000% | 800.00 |

### Example 43

| Ingredient | % w/w | mg/tab |
|---|---|---|
| Hydrocodone Bitartrate | 1.110% | 9.99 |
| APAP (96.4%) | 37.460% | 337.14 |
| Mannitol | 11.530% | 103.77 |
| Carbopol | 5.560% | 50.04 |
| MCC | 15.000% | 135.00 |
| Crospovidone | 25.000% | 225.00 |
| Sodium Bicarbonate | 3.340% | 30.06 |
| Magnesium Stearate | 1.000% | 9.00 |
| Total | 100.000% | 900.00 |

### Example 44

| Ingredient | % w/w | mg/tab |
|---|---|---|
| Hydrocodone Bitartrate | 1.000% | 10.00 |
| APAP (96.4%) | 33.710% | 337.10 |
| Mannitol | 6.290% | 62.90 |
| Carbopol | 5.000% | 50.00 |
| MCC | 30.000% | 300.00 |
| Crospovidone | 20.000% | 200.00 |
| Sodium Bicarbonate | 3.000% | 30.00 |
| Magnesium Stearate | 1.000% | 10.00 |
| Total | 100.000% | 1000.00 |

### Example 45

| Ingredient | % w/w | mg/tab |
|---|---|---|
| Hydrocodone Bitartrate | 1.429% | 10.00 |
| APAP (96.4%) | 48.162% | 337.13 |
| Mannitol | 5.266% | 36.86 |
| Carbopol | 14.286% | 100.00 |
| MCC | 8.000% | 56.00 |
| Crospovidone | 15.000% | 105.00 |
| Sodium Bicarbonate | 6.857% | 48.00 |
| Magnesium Stearate | 1.000% | 7.00 |
| Total | 100.000% | 700.00 |

1. An immediate release compressed oral dosage form comprising:
an active pharmaceutical ingredient,
from 2.5 to 35 weight percent gelling polymer comprising a carbomer polymer,
from 15 to 35 weight percent disintegrant,
from 3 to 80 weight percent filler, and
a pH adjuster,
the weight percent amounts being based on a total weight of the dosage form.

2. The dosage form according to claim 1 comprising from 3 to 32 weight percent of the gelling polymer.

3. The dosage form according to claim 1 comprising filler selected from mannitol, microcrystalline cellulose, and combinations thereof.

4. The dosage form according to claim 3 comprising
from 6 to 80 weight percent microcrystalline cellulose, and
from 1 to 60 weight percent mannitol,
based on total weight dosage form.

5. The dosage form according to claim 3 comprising
from 15 to 50 weight percent microcrystalline cellulose, and
from 1 to 15 weight percent mannitol,
based on total weight dosage form.

6. The dosage form according to claim 1, comprising from 0.5 to 50 weight percent pH-adjuster.

7. The dosage form according to claim 1, comprising from 1 to 8 weight percent pH-adjuster.

8. The dosage form according to claim 1, wherein the pH adjuster is sodium bicarbonate.

9. The dosage form according to claim 1, wherein the disintegrant is selected from the group consisting of: sodium starch glycolate, croscarmellose sodium, corn starch, crospovidone, and combinations thereof.

10. The dosage form according to claim 1, comprising
from 2.5 to 35 percent of the carbomer polymer
from 1 to 8 weight percent sodium bicarbonate as a pH-adjuster,
from 12 to 40 weight percent disintegrant,
from 6 to 30 weight percent microcrystalline cellulose as filler,
from 1 to 15 weight percent mannitol as filler, and
an active pharmaceutical ingredient,
the weight percents being based on a total weight of the dosage form.

11. An immediate release compressed oral dosage form comprising:
an active pharmaceutical ingredient,
from 1 to 20 weight percent gelling polymer comprising xanthan gum,
from 15 to 35 weight percent disintegrant, and
from 3 to 80 weight percent filler,
the weight percent amounts being based on a total weight of the dosage form.

12. The dosage form according to claim 11 comprising from 2 to 15 weight percent of the gelling polymer.

13. The dosage form according to claim 11 comprising filler selected from mannitol, microcrystalline cellulose, and combinations thereof.

14. The dosage form according to claim 13 comprising
from 6 to 80 weight percent microcrystalline cellulose, and
from 1 to 60 weight percent mannitol,
based on total weight dosage form.

15. The dosage form according to claim 13 comprising
from 15 to 50 weight percent microcrystalline cellulose, and
from 1 to 15 weight percent mannitol,
based on total weight dosage form.

16. The dosage form according to claim 11, wherein the disintegrant is selected from the group consisting of: sodium starch glycolate, starch, croscarmellose sodium, corn starch, crospovidone, and combinations thereof.

17. The dosage form according to claim 11 comprising
from 2.5 to 35 percent of the xanthan gum,
from 12 to 40 weight percent disintegrant,
from 6 to 30 weight percent microcrystalline cellulose as filler,
from 1 to 15 weight percent mannitol as filler, and
the active pharmaceutical ingredient,
the weight percents being based on a total weight of the dosage form.

18. The dosage form according to claim 1, wherein the dosage form excludes an emetic, a nasal irritant, and an effervescent.

19. The dosage form according to claim 1, wherein the active pharmaceutical ingredient is an opioid and the dosage form excludes an opioid antagonist.

20. The dosage form according to claim 1, wherein the dosage form releases at least 80 percent of the active pharmaceutical ingredient within 3 hours of ingestion by a human.

21. The dosage form according to claim 1, wherein the active pharmaceutical ingredient is one that is commonly susceptible to abuse.

22. The dosage form according to claim 1, wherein the active pharmaceutical ingredient is selected from the group consisting of a sedative hypnotic, a stimulant, a depressant, an anxiolytic, and a narcotic analgesic.

23. The dosage form according to claim 1, wherein the active pharmaceutical ingredient is selected from the group consisting of an opioid, a barbiturate, a benzodiadepine, and an amphetamine.

24. The dosage form according to claim 1, wherein the active pharmaceutical ingredient is an opioid.

25. The dosage form according to claim 1, wherein the active pharmaceutical ingredient is selected from the group consisting of buprenorphine, codeine, dihydrocodeine, dihydromorphine, hydrocodone, hydromorphone, pseudoephedrine, morphine, oxycodone, oxymorphone, and pharmaceutically acceptable salts therefore.

26. The dosage form according to claim 1, comprising a non-steroidal analgesic drug.

27. The dosage form according to claim 1, comprising from 10 to 75 weight percent of the non-steroidal analgesic drug based on a total weight of the dosage form.

28. The dosage form according to claim 1, wherein the dosage form is in a compressed capsule or a compressed tablet form.

29. The dosage form according to claim 1, wherein the dosage form inhibits isolation of a solution of the API using a syringe, at 25 degrees Celsius, upon grinding the dosage form and combining the dosage form with 10 milliliters of water.

30. The dosage form according to claim 1, wherein the dosage form inhibits isolation of a solution of the API using a syringe, at 100 degrees Celsius, upon grinding the dosage form and combining the dosage form with 10 milliliters of water.

31. A method of preparing an immediate release dosage form according to claim 1, the method comprising providing ingredients comprising:
the active pharmaceutical ingredient,
the gelling polymer,
the disintegrant, and
the filler, and
compressing the ingredients into a compressed immediate release dosage form.

32. A method of administering an active pharmaceutical agent to a subject in need thereof, the method comprising,
providing a dosage form according to claim 1, and
administering the dosage form to the subject.

33. A method of preventing, alleviating, or ameliorating a level of pain in a subject, comprising administering to the subject a dosage form as recited in claim 24.

34. A method of preventing, alleviating, or ameliorating a level of pain in a subject, comprising administering to the subject a dosage form as recited in claim 25.

35. The dosage form according to claim 11, wherein the dosage form excludes an emetic, a nasal irritant, and an effervescent.

36. The dosage form according to claim 11, wherein the active pharmaceutical ingredient is an opioid and the dosage form excludes an opioid antagonist.

37. The dosage form according to claim 11, wherein the dosage form releases at least 80 percent of the active pharmaceutical ingredient within 3 hours of ingestion by a human.

38. The dosage form according to claim 11, wherein the active pharmaceutical ingredient is one that is commonly susceptible to abuse.

39. The dosage form according to claim 11, wherein the active pharmaceutical ingredient is selected from the group consisting of a sedative hypnotic, a stimulant, a depressant, an anxiolytic, and a narcotic analgesic.

40. The dosage form according to claim 11, wherein the active pharmaceutical ingredient is selected from the group consisting of an opioid, a barbiturate, a benzodiadepine, and an amphetamine.

41. The dosage form according to claim 11, wherein the active pharmaceutical ingredient is an opioid.

42. The dosage form according to claim 11, wherein the active pharmaceutical ingredient is selected from the group consisting of buprenorphine, codeine, dihydrocodeine, dihydromorphine, hydrocodone, hydromorphone, pseudoephedrine, morphine, oxycodone, oxymorphone, and pharmaceutically acceptable salts therefore.

43. The dosage form according to claim 11, comprising a non-steroidal analgesic drug.

44. The dosage form according to claim 11, comprising from 10 to 75 weight percent of the non-steroidal analgesic drug based on a total weight of the dosage form.

45. The dosage form according to claim 11, wherein the dosage form is in a compressed capsule or a compressed tablet form.

46. The dosage form according to claim 11, wherein the dosage form inhibits isolation of a solution of the API using a syringe, at 25 degrees Celsius, upon grinding the dosage form and combining the dosage form with 10 milliliters of water.

47. The dosage form according to claim 11, wherein the dosage form inhibits isolation of a solution of the API using a syringe, at 100 degrees Celsius, upon grinding the dosage form and combining the dosage form with 10 milliliters of water.

48. A method of preparing an immediate release dosage form according to claim 11 comprising providing ingredients comprising:
the active pharmaceutical ingredient,
the gelling polymer,
the disintegrant, and
the filler, and
compressing the ingredients into a compressed immediate release dosage form.

49. A method of administering an active pharmaceutical agent to a subject in need thereof, the method comprising,
providing a dosage form according to claim 11, and
administering the dosage form to the subject.
